(19) United States
(12) Patent Application Publication
(10) Pub. No.: US 2026/0191446 A1
(43) Pub. Date: Jul. 9, 2026

(54) PERSONALITY ESTIMATION DEVICE, METHOD AND PROGRAM

(71) Applicant: NTT, Inc., Tokyo (JP)

(72) Inventors: Jumpei YAMASHITA, Musashino-shi (JP); Hiroshi YOSHIDA, Musashino-shi (JP); Hidetaka KOYA, Musashino-shi (JP)

(73) Assignee: NTT, Inc., Tokyo (JP)

(21) Appl. No.: 19/132,392

(22) PCT Filed: Nov. 29, 2022
(86) PCT No.: PCT/JP2022/043944

§ 371 Date: May 23, 2025

(51) Int. Cl. A61B 5/16

(52) U.S. Cl. CPC A61B 5/163

(57) ABSTRACT

According to an embodiment, a personality estimation device includes a first, second, third, fourth, fifth, and sixth units. The first and second units acquire and store first and second relationship information indicating a relationship between personality and eyeball movement of a person. The third unit acquires information indicating an eye closing-related index of a target person. The fourth unit determines whether a current situation of the target person is the first situation or the second situation on a basis of the acquired information, and selects one of the first relationship information and the second relationship information. The fifth unit acquires information indicating movement of an eyeball or a head of the target person. The sixth unit estimates a personality of the target person on a basis of the acquired information indicating the movement of the eyeball or the head and the selected first relationship information or second relationship information.

### TECHNICAL FIELD

One aspect of the present invention relates to a personality estimation device, a method, and a program for estimating a human personality, for example.

### BACKGROUND ART

Capturing human traits is important because it leads to estimation of psychological attributes related to individual performance in various fields.

For example, in the Big Five theory, which expresses a human personality by five latent factors (traits), it is known that five traits of openness, conscientiousness, extraversion, agreeableness, and neuroticism are commonly present in humans, and a personality is expressed by a combination of specific characteristic values, such as a specific individual having “high openness and low conscientiousness . . . ” (see, for example, Non Patent Literature 1).

In addition, it is also known that patterns of thought, emotion, and behavior of each individual are different for each personality, and the personality affects a wide range of psychological attributes of a person such as a preference for an occupation. For example, openness is associated with a preference for scholarly and artistic occupations. For this reason, for example, estimating the openness is expected to be used to predict a preference for work in a company.

Typically, a personality is estimated from answers to a questionnaire (questionnaire) or the like. However, in particular, in a case where a personality is estimated for the purpose of determining a preference for work, there is a possibility that the estimation is inaccurate because answers of a person include a bias to show the person in a desirable form.

Therefore, it is considered to estimate a personality from eyeball movement in normal life. However, the relationship between personality and eyeball movement varies depending on the situation in which an estimation target person is placed. For example, when the degree of openness in personality or the degree of curiosity, which is deeply related to the openness, is higher, frequent eyeball movement occurs in a situation of viewing a scene. Meanwhile, when the degree of openness is higher, the occurrence frequency of the eyeball movement decreases in a situation of viewing an abstract moving image. These traits are each known from Non Patent Literatures 2 and 3.

### CITATION LIST

### Non Patent Literature

Non Patent Literature 1: Larson, L. M., Rottinghaus, P. J., & Borgen, F. H. (2002). “Meta-analyses of Big Six interests and Big Five personality factors”. Journal of Vocational Behavior, 61(2), 217-239.
Non Patent Literature 2: Risko, E. F., Anderson, N. C., Lanthier, S., & Kingstone, A. (2012). “Curious eyes: Individual differences in personality predict eye movement behavior in scene-viewing”. Cognition, 122(1), 86-90.
Non Patent Literature 3: Rauthmann, J. F., Seubert, C. T., Sachse, P., & Furtner, M. R. (2012). “Eyes as windows to the soul: Gazing behavior is related to personality”. Journal of Research in Personality, 46(2), 147-156.

### SUMMARY OF INVENTION

### Technical Problem

As described above, the relationship between personality and eyeball movement of a person varies depending on the situation in which an estimation target person is placed. For this reason, it is difficult to comprehensively collect the relationship between personality and eyeball movement for every situation appearing in normal life. In addition, even if a comprehensive relationship list is obtained, it is difficult to determine the situation in which the estimation target person is currently placed without fail.

The present invention has been made in view of the above circumstances, and aims to provide a technique that makes it possible to easily and effectively estimate a personality of a person in estimation of the personality of the person based on of the relationship between personality and eyeball movement, only by giving a certain definition of a situation, rather than considering every situation in which the person is placed.

### Solution to Problem

In order to solve the above problems, one aspect of a personality estimation device or an estimation method according to the present invention acquires and stores the first relationship information indicating the relationship between personality and eyeball movement of a person, which is created in advance in correspondence with the first situation in which the person focuses on processing of visual external information, and acquires and stores the second relationship information indicating the relationship between personality and eyeball movement of the person, which is created in advance in correspondence with the second situation in which the person focuses on processing of visual internal information. In this state, first, information indicating an eye closing-related index of an estimation target person US is acquired, the current situation of the estimation target person is determined on the basis of the information indicating the eye closing-related index, and the relationship information corresponding to the determined current situation is selected. Next, information indicating movement of an eyeball or the head of the estimation target person is acquired, a personality of the estimation target person is estimated on the basis of the acquired information indicating the movement of the eyeball or head and the selected first relationship information or second relationship information, and information indicating the estimation result is output.

According to one aspect of the present invention, only two types of situations, that is, the first situation focusing on processing of visual external information and the second situation focusing on processing of visual internal information are defined as main situations that occur in behavior of a person, and information indicating the relationship between personality and eyeball movement of a person corresponding to these situations is prepared. The personality of the estimation target person is then estimated on the basis of the determination result of the situation of the estimation target person and the information indicating the relationship between personality and eyeball movement corresponding to the situation. Therefore, it is not necessary to comprehensively prepare the relationship information between personality and eyeball movement in correspondence with every situation of the estimation target person. In addition, since the situation of the estimation target person is determined on the basis of the eye closing-related index, it is possible to easily determine the situation of the estimation target person.

### Advantageous Effects of Invention

That is, according to one aspect of the present invention, it is possible to provide a technique that makes it possible to easily and effectively estimate a personality of a person in estimation of the personality of the person based on of the relationship between personality and eyeball movement, only by giving a certain definition of a situation, rather than considering every situation in which the person is placed.

### BRIEF DESCRIPTION OF DRAWINGS

FIG. 1 is a block diagram illustrating an example of a hardware configuration of a personality estimation device according to one embodiment of the present invention.

FIG. 2 is a block diagram illustrating an example of a software configuration of the personality estimation device according to the one embodiment of the present invention.

FIG. 3 is a flowchart illustrating an example of a processing procedure and processing contents of personality estimation processing executed by a control unit of the personality estimation device illustrated in FIG. 2.

### DESCRIPTION OF EMBODIMENTS

Hereinafter, an embodiment according to the present invention will be described with reference to the drawings.

### One Embodiment

### (Overview)

There are two types of representative situations that appear in a user engaged in some work: a situation that focuses on processing of visual external information and a situation that focuses on processing of visual internal information.

Focusing on the above point, one embodiment of the present invention registers in advance two sets of relationship information each of which indicates the relationship between personality and eyeball movement of a person and corresponds to one of the two types of situations in a personality estimation device. In this state, first, an eye closing-related index of an estimation target person is acquired, the current situation of the estimation target person is determined on the basis of the eye closing-related index, and the relationship information corresponding to the determined situation is selected. Next, information indicating movement of an eyeball or the head of the estimation target person is acquired, and a personality of the estimation target person is estimated on the basis of the acquired information indicating the movement of the eyeball or the head and the selected relationship information.

As a result, it is sufficient to prepare only two sets of relationship information corresponding to the two types of situations as the information indicating the relationship between personality and eyeball movement, and thus, it is not necessary to perform an operation of defining every situation of a human and comprehensively preparing relationship information between personality and eyeball movement corresponding to all the situations. In addition, since the current situation of the estimation target person is determined on the basis of the eye closing-related index, it is possible to accurately and easily determine the current situation of the estimation target person.

### Configuration Example

A personality estimation device according to the one embodiment of the present invention includes, for example, a personal computer used by an estimation target person or the like. Note that, as the personality estimation device, a server computer disposed on a local area network (LAN), the web, or a cloud may be used in addition to the personal computer.

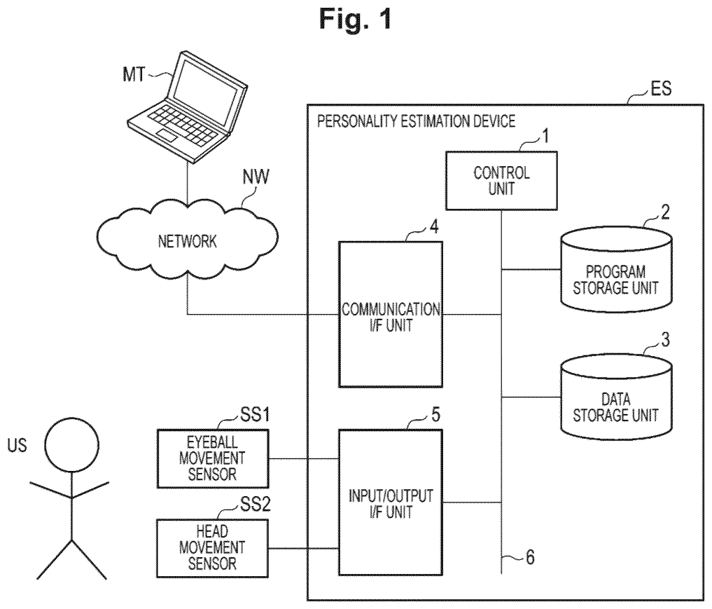

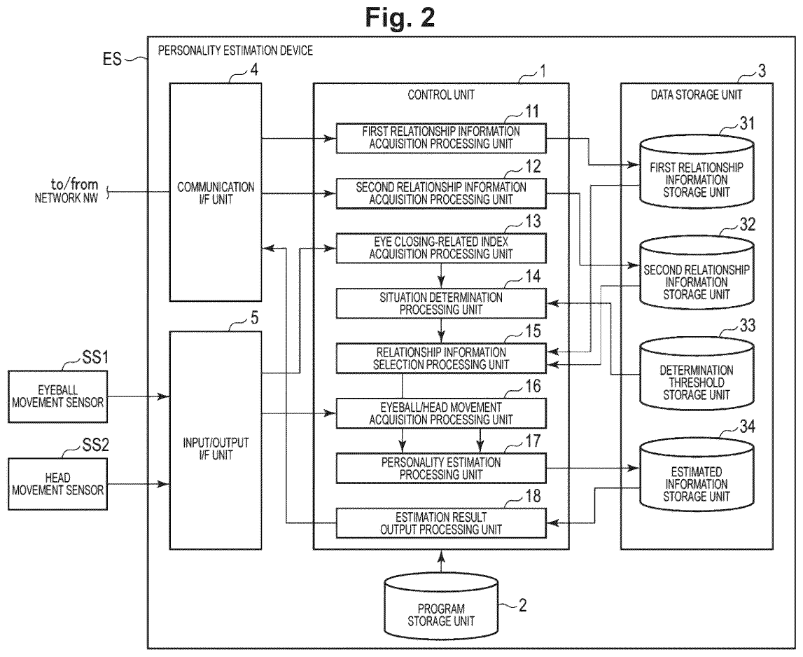

FIG. 1 is a block diagram illustrating an example of a hardware configuration of the personality estimation device according to the one embodiment of the present invention, and FIG. 2 is a block diagram illustrating an example of a software configuration of the personality estimation device according to the one embodiment of the present invention.

A personality estimation device ES includes a control unit 1 using a hardware processor such as a central processing unit (CPU), and a storage unit including a program storage unit 2 and a data storage unit 3, a communication interface (hereinafter, an interface is abbreviated as an I/F) unit 4, and an input/output I/F unit 5 are connected to the control unit via a bus 6.

Under the control of the control unit 1, the communication I/F unit 4 transmits and receives information data to and from an administrator terminal MT used by, for example, a system administrator or the like in accordance with a communication protocol defined by a network NW.

The administrator terminal MT is used to register in advance information indicating the relationship between personality and eyeball movement of a person in the personality estimation device ES. As described above, the information indicating the relationship between personality and eyeball movement of a person is created in correspondence with each of the first situation focusing on processing of visual external information and the second situation focusing on processing of visual internal information. An example of a method of creating the relationship information will be described in Operation Example.

The input/output I/F unit 5 is connected to an eyeball movement sensor SS1 and a head movement sensor SS2 attached to the estimation target person US.

For example, an eye electrometer is used as the eyeball movement sensor SS1. For example, the eye electrometer attaches an electrode to a peripheral portion of an eyeball, detects blinking motion of the eyeball on the basis of a change in potential detected by the electrode, and outputs a detection signal thereof.

The head movement sensor SS2 includes, for example, an acceleration sensor or an angular velocity sensor, and is mounted on the head of the estimation target person US. The head movement sensor SS2 detects movement of the head of the estimation target person US, and outputs a detection signal thereof.

The input/output I/F unit 5 receives detection signals output from the eyeball movement sensor SS1 and the head movement sensor SS2, and converts the detection signals into digital data that can be handled by the control unit 1.

Note that, as the eyeball movement sensor SS1 and the head movement sensor SS2, for example, a sensor that detects eyeball movement or head movement by capturing an image of a range including the eyeball site or the head with a camera and analyzing the captured image may be used.

The program storage unit 2 is configured by, for example, combining a nonvolatile memory on which writing and reading can be performed as necessary, such as a solid state drive (SSD), and a nonvolatile memory such as a read only memory (ROM) as storage media, and stores application programs necessary for executing various types of control processing according to the one embodiment, in addition to middleware such as an operating system (OS). Note that, hereinafter, the OS and the application programs will be collectively referred to as a program.

The data storage unit 3 is a combination of, for example, a nonvolatile memory on which writing and reading can be performed as necessary, such as an SSD, and a volatile memory such as a random access memory (RAM) as storage media, and a storage area of the data storage unit 3 includes a first relationship information storage unit 31, a second relationship information storage unit 32, a determination threshold storage unit 33, and an estimated information storage unit 34 as main storage units necessary for implementing the one embodiment of the present invention.

The first relationship information storage unit 31 and the second relationship information storage unit 32 are respectively used to store the first and second relationship information transmitted from the administrator terminal MT and indicating the relationship between personality and eyeball movement.

The determination threshold storage unit 33 stores a threshold used to determine the situation of the estimation target person US on the basis of an eye closing-related index.

The estimated information storage unit 34 is used to temporarily store information indicating an estimation result of a personality estimated for the estimation target person US.

The control unit 1 includes a first relationship information acquisition processing unit 11, a second relationship information acquisition processing unit 12, an eye closing-related index acquisition processing unit 13, a situation determination processing unit 14, a relationship information selection processing unit 15, an eyeball/head movement acquisition processing unit 16, a personality estimation processing unit 17, and an estimation result output processing unit 18 as processing functions necessary for implementing the one embodiment of the present invention.

Each of the above-described processing units 11 to 18 of the control unit 1 is implemented by causing the hardware processor of the control unit 1 to execute an application program stored in the program storage unit 2. Note that some or all of the processing units 11 to 18 may be implemented with hardware such as a large scale integration (LSI) or an application specific integrated circuit (ASIC).

The first relationship information acquisition processing unit 11 receives, via the communication I/F unit 4, the first relationship information transmitted from the administrator terminal MT and indicating the relationship between personality and eyeball movement corresponding to the first situation, and stores the received first relationship information in the first relationship information storage unit 31.

Similarly to the first relationship information acquisition processing unit 11, the second relationship information acquisition processing unit 12 also receives, via the communication I/F unit 4, the second relationship information transmitted from the administrator terminal MT and indicating the relationship between personality and eyeball movement corresponding to the second situation, and stores the received second relationship information in the second relationship information storage unit 32.

The eye closing-related index acquisition processing unit 13 performs processing of capturing a detection signal of eyeball movement output from the eyeball movement sensor SS1 attached to the estimation target person US via the input/output I/F unit 5, and obtaining the eye closing-related index of the estimation target person US on the basis of digital data of the captured detection signal.

The situation determination processing unit 14 compares the eye closing-related index obtained by the eye closing-related index acquisition processing unit 13 with the determination threshold stored in the determination threshold storage unit 33 to determine whether the current situation of the estimation target person US is the first situation focusing on processing of visual external information or the second situation focusing on processing of visual internal information.

The relationship information selection processing unit 15 selects one of the first relationship information corresponding to the determined first situation or the second relationship information corresponding to the second situation from the first or second relationship information storage unit 31 or 32 on the basis of the determination result of the situation determination processing unit 14.

The eyeball/head movement acquisition processing unit 16 acquires a detection signal representing eyeball movement or a detection signal representing head movement of the estimation target person US from the eyeball movement sensor SS1 or the head movement sensor SS2 via the input/output I/F unit 5, and calculates the movement frequency representing the temporal periodicity of the eyeball movement or the head movement on the basis of the acquired detection signal.

The personality estimation processing unit 17 estimates a personality corresponding to the frequency of the eyeball movement or the head movement from the first or second relationship information on the basis of the frequency of the eyeball movement or the head movement obtained by the eyeball/head movement acquisition processing unit 16 and the first or second relationship information selected by the relationship information selection processing unit 15. The personality estimation processing unit 17 then stores information indicating the estimation result in the estimated information storage unit 34.

The estimation result output processing unit 18 reads the information indicating the estimation result of the personality from the estimated information storage unit 34, and transmits the read information from the communication I/F unit 4 to, for example, the administrator terminal MT.

### Operation Example

Next, an operation example of the personality estimation device ES configured as described above will be described.

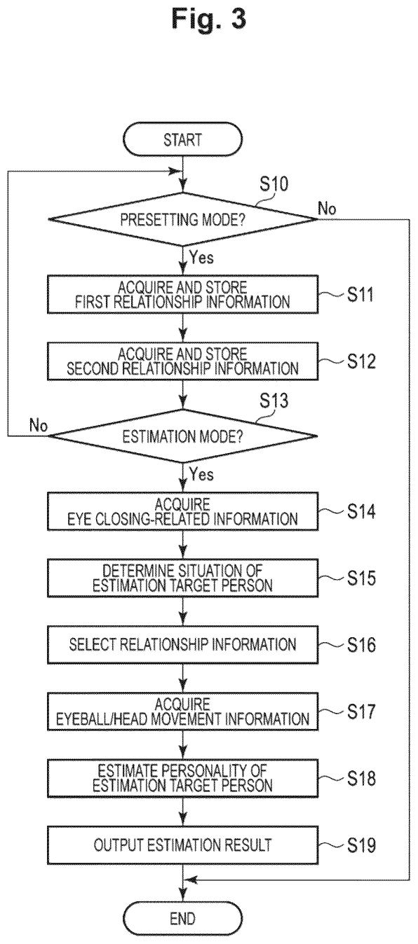

FIG. 3 is a flowchart illustrating an example of a processing procedure and processing contents of personality estimation processing executed by the control unit 1 of the personality estimation device ES.

### (1) Presetting of Relationship Information

Information indicating the relationship between personality and eyeball movement is created in advance by the administrator, for example. As described above, as the relationship information, two types relationship information are created: the first relationship information corresponding to the first situation focusing on processing of visual external information and the second relationship information corresponding to the second situation focusing on processing of visual internal information.

First, for example, a questionnaire survey is conducted on a plurality of workers selected as experiment targets, and the personality traits of each worker are determined on the basis of their answers. In this embodiment, “openness”, which is one personality, is determined. Note that the number of workers is set to, for example, several tens to several hundreds, but the value is arbitrary.

Next, the plurality of workers is caused to perform typical desk work such as document transfer work. During the work period, eyeball movement of each worker is measured with, for example, an eye electrometer. Note that, instead of the eyeball movement, movement of the head of each worker during the work period may be measured with, for example, an acceleration sensor or an angular velocity sensor, and the eyeball movement or the head movement may be measured by a method of detecting the position of an iris on the basis of a camera image.

As a result of this measurement, it can be seen that a worker with higher openness tends to have a higher frequency of the eyeball movement, that is, tends to have a shorter cycle of the eyeball movement during the period in which the worker is performing the typical desk work, that is, in the situation that focuses on processing of visual external information.

Meanwhile, the plurality of workers is caused to perform work such as technical research or writing of technical materials, which is atypical desk work, and eyeball movement or head movement of each worker during the work period is measured as in the case of the qualitative desk work. As a result of this measurement, it can be seen that a worker with higher openness tends to have a lower frequency of the eyeball movement, that is, tends to have a longer cycle of the eyeball movement during the period in which the worker is performing the atypical desk work, that is, in the situation that focuses on processing of visual internal information.

On the basis of the above measurement results, the administrator creates each of the first relationship information indicating the relationship between personality and eyeball movement corresponding to the first situation focusing on processing of visual external information and the second relationship information indicating the relationship between personality and eyeball movement corresponding to the second situation focusing on processing of visual internal information.

The administrator then transmits the created first relationship information and second relationship information from the administrator terminal MT to the personality estimation device ES.

Meanwhile, when the first and second relationship information are transmitted from the administrator terminal MT in a state where a presetting mode is set, the control unit 1 of the personality estimation device ES proceeds from step S10 to steps S11 and S12. Under the control of the first relationship information acquisition processing unit 11 and the second relationship information acquisition processing unit 12, each of the first and second relationship information transmitted from the administrator terminal MT is received via the communication I/F unit 4, and the received first and second relationship information are stored in the first and second relationship information storage units 31 and 32, respectively.

Note that, in the above description, a case where the first and second relationship information are created in the administrator terminal MT and registered in the personality estimation device ES has been described as an example. However, for example, the personality estimation device ES may download and register the relationship information stored in advance in a server computer or the like. Furthermore, the creation processing of the relationship information may be performed in the personality estimation device ES.

### (2) Estimation of Personality

### (2-1) Determination of Situation of Estimation Target Person US

In a case where a personality of the estimation target person US is determined, the eyeball movement sensor SS1 and the head movement sensor SS2 are attached to the head of the estimation target person US during a work period. Note that the head movement sensor SS2 is not necessarily mounted.

Meanwhile, when a personality estimation mode is set, the control unit 1 of the personality estimation device ES detects the setting of the estimation mode in step S13. First, under the control of the eye closing-related index acquisition processing unit 13, in step S14, the control unit 1 of the personality estimation device ES captures a detection signal output from the eyeball movement sensor SS1 via the input/output I/F unit 5, and obtains eye closing-related information of the estimation target person US. For example, the number of blinks per unit time is counted on the basis of the detection signal of the eyeball movement, and an average blinking frequency of the estimation target person US is calculated on the basis of the count value.

Subsequently, under the control of the situation determination processing unit 14, in step S15, the control unit 1 of the personality estimation device ES compares the calculated average blinking frequency of the estimation target person US with a determination threshold stored in the determination threshold storage unit 33. As a result of the comparison, when the average blinking frequency is lower than the threshold, it is determined that the estimation target person US is in the first situation focusing on processing of visual external information. On the other hand, as a result of the comparison, when the average blinking frequency is equal to or higher than the threshold, it is determined that the estimation target person US is in the second situation focusing on processing of visual internal information.

### (2-2) Selection of Relationship Information

Next, under the control of the relationship information selection processing unit 15, in step S16, the control unit 1 of the personality estimation device ES selectively reads the first or second relationship information corresponding to the current situation of the estimation target person US from the first or second relationship information storage unit 31 or 32 on the basis of the determination result of the situation of the estimation target person US. The relationship information selection processing unit 15 then passes the read first or second relationship information to the personality estimation processing unit 17.

### (2-3) Estimation and Output of Personality of Estimation Target Person

First, under the control of the eyeball/head movement acquisition processing unit 16, in step S17, the control unit 1 of the personality estimation device ES captures a detection signal transmitted from the eyeball movement sensor SS1 attached to the head of the estimation target person US as digital data via the input/output I/F unit 5. On the basis of the captured digital data of the detection signal, the eyeball/head movement acquisition processing unit 16 then calculates, for example, the number of occurrences of the eyeball movement per unit time, that is, the average frequency of the eyeball movement. The calculated frequency of the eyeball movement is then passed to the personality estimation processing unit 17.

Note that, instead of calculating the frequency of the eyeball movement, a detection signal representing the head movement may be acquired from the head movement sensor SS2, and the frequency of the head movement of the estimation target person US may be obtained from the acquired detection signal of the head movement.

Next, under the control of the personality estimation processing unit 17, in step S18, the control unit 1 of the personality estimation device ES determines a personality corresponding to the frequency of the eyeball movement or the head movement from the first or second relationship information on the basis of the frequency of the eyeball movement or the head movement obtained by the eyeball/head movement acquisition processing unit 16 and the first or second relationship information selected by the relationship information selection processing unit 15. In this example, “whether the openness is high or low” is determined as the personality. The personality estimation processing unit 17 then stores the determination result of the personality in the estimated information storage unit 34 as information indicating the estimation result of the personality of the estimation target person US.

Note that, as the personality to be estimated, in addition to the “openness”, conscientiousness, extraversion, agreeableness, neuroticism, or the like may be estimated.

Finally, under the control of the estimation result output processing unit 18, in step S19, the control unit 1 of the personality estimation device ES reads the information indicating the estimation result of the personality of the estimation target person US from the estimated information storage unit 34, and transmits the read information indicating the estimation result from the communication I/F unit 4 to the administrator terminal MT.

Note that the information indicating the estimation result of the personality may be transmitted to a terminal used by the estimation target person.

### (Actions and Effects)

As described above, the one embodiment acquires each of the first relationship information indicating the relationship between personality and eyeball movement of a person, which is created in advance in correspondence with the first situation in which the person focuses on processing of visual external information, and the second relationship information indicating the relationship between personality and eyeball movement of the person, which is created in advance in correspondence with the second situation in which the person focuses on processing of visual internal information, and sores the first and second relationship information in the first and second relationship information storage units 31 and 32. In this state, first, information indicating an eye closing-related index of the estimation target person US is acquired, the current situation of the estimation target person US is determined on the basis of the information indicating the eye closing-related index, and the relationship information corresponding to the current situation is selected. Next, a detection signal of eyeball movement or head movement of the estimation target person US is acquired, a personality of the estimation target person US is estimated on the basis of the acquired detection signal of the eyeball movement or the head movement and the selected first or second relationship information, and information indicating the estimation result is output.

Accordingly, it is sufficient to prepare only two sets of relationship information corresponding to the two types of situations as the information indicating the relationship between personality and eyeball movement, and thus, it is not necessary to perform an operation of defining every situation of the estimation target person US and comprehensively preparing relationship information between personality and eyeball movement in correspondence with all the situations. In addition, since the situation of the estimation target person US is determined on the basis of the eye closing-related index, it is possible to easily determine the situation of the estimation target person US.

### OTHER EMBODIMENTS

In the one embodiment, a case where the processing functions from the relationship information acquisition processing units 11 and 12 to the estimation result output processing unit 18 are provided in a single personal computer has been described as an example. However, the processing functions may be distributed to a plurality of personal computers or server computers.

In addition, the method of acquiring the first and second relationship information, the type of the eye closing-related index of the estimation target person, the method of acquiring the eye closing-related index, the contents of the processing of determining the situation of the estimation target person using the eye closing-related index, the configuration of the information indicating the eyeball movement or the head movement of the estimation target person, the method of acquiring the information, the contents of the processing of determining the personality, and the like can be variously modified and implemented without departing from the gist of the invention.

Although the embodiments of the present invention have been described in detail above, the above description is merely an example of the present invention in all respects. It is needless to say that various improvements and modifications can be made without departing from the scope of the present invention. That is, in implementing the present invention, a specific configuration according to the embodiment may be appropriately adopted.

In short, the present invention is not limited to the above embodiments without any change, but can be embodied by modifying the components without departing from the gist of the invention at the implementation stage. Furthermore, various inventions can be formed by appropriately combining a plurality of components disclosed in the above embodiments. For example, some components may be deleted from all the components shown in the embodiments. Furthermore, components in different embodiments may be appropriately combined.

### REFERENCE SIGNS LIST

ES Personality estimation device
US Estimation target person
MT Administrator terminal
NW Network
SS1 Eyeball movement sensor
SS2 Head movement sensor
1 Control unit
2 Program storage unit
3 Data storage unit
4 Communication I/F unit
5 Input/output I/F unit
6 Bus
11 First relationship information acquisition processing unit
12 Second relationship information acquisition processing unit
13 Eye closing-related index acquisition processing unit
14 Situation determination processing unit
15 Relationship information selection processing unit
16 Eyeball/head movement acquisition processing unit
17 Personality estimation processing unit
18 Estimation result output processing unit
31 First relationship information storage unit
32 Second relationship information storage unit
33 Determination threshold storage unit
34 Estimated information storage unit

1. A personality estimation device comprising:
processing circuitry configured to:
acquire and store first relationship information indicating a relationship between personality and eyeball movement of a person, the first relationship information being created in advance in correspondence with a first situation in which the person focuses on processing of visual external information;
acquire and store second relationship information indicating a relationship between personality and eyeball movement of the person, the second relationship information being created in advance in correspondence with a second situation in which the person focuses on processing of visual internal information;
acquire information indicating an eye closing-related index of an estimation target person;
determine whether a current situation of the estimation target person is the first situation or the second situation on a basis of the acquired information indicating the eye closing-related index, and select one of the first relationship information and the second relationship information on a basis of a determination result;
acquire information indicating movement of an eyeball or a head of the estimation target person;
estimate a personality of the estimation target person on a basis of the acquired information indicating the movement of the eyeball or the head and the selected first relationship information or second relationship information; and
output information indicating an estimation result of the personality of the estimation target person.

2. The personality estimation device according to claim 1, wherein:
the processing circuitry is configured to acquire measurement information indicating at least one of an eye potential of the estimation target person and a captured image including an eyeball site, and obtain the eye closing-related index of the estimation target person on a basis of the acquired measurement information.

3. The personality estimation device according to claim 1, wherein:
the processing circuitry is configured to acquire an average blinking frequency of the estimation target person as the eye closing-related index.

4. The personality estimation device according to claim 1, wherein;
the processing circuitry is configured to acquire information indicating a frequency of the movement of the eyeball or the head as the information indicating the movement of the eyeball or the head of the estimation target person.

5. The personality estimation device according to claim 1, wherein:
the processing circuitry is configured to estimate openness as the personality of the estimation target person.

6. A personality estimation method, comprising:
acquiring and storing first relationship information indicating a relationship between personality and eyeball movement of a person, the first relationship information being created in advance in correspondence with a first situation in which the person focuses on processing of visual external information;
acquiring and storing second relationship information indicating a relationship between personality and eyeball movement of the person, the second relationship information being created in advance in correspondence with a second situation in which the person focuses on processing of visual internal information;
acquiring information indicating an eye closing-related index of an estimation target person;
determining whether a current situation of the estimation target person is the first situation or the second situation on a basis of the acquired information indicating the eye closing-related index, and selecting one of the first relationship information and the second relationship information on a basis of a determination result;
acquiring information indicating movement of an eyeball or a head of the estimation target person;
estimating a personality of the estimation target person on a basis of the acquired information indicating the movement of the eyeball or the head and the selected first relationship information or second relationship information; and
outputting information indicating an estimation result of the personality of the estimation target person.

7. A non-transitory computer-readable storage medium including instructions executable by one or more processors to cause the one or more processors to perform operations comprising:
acquiring and storing first relationship information indicating a relationship between personality and eyeball movement of a person, the first relationship information being created in advance in correspondence with a first situation in which the person focuses on processing of visual external information;
acquiring and storing second relationship information indicating a relationship between personality and eyeball movement of the person, the second relationship information being created in advance in correspondence with a second situation in which the person focuses on processing of visual internal information;
acquiring information indicating an eye closing-related index of an estimation target person;
determining whether a current situation of the estimation target person is the first situation or the second situation on a basis of the acquired information indicating the eye closing-related index, and selecting one of the first relationship information and the second relationship information on a basis of a determination result;
acquiring information indicating movement of an eyeball or a head of the estimation target person;
estimating a personality of the estimation target person on a basis of the acquired information indicating the movement of the eyeball or the head and the selected first relationship information or second relationship information; and
outputting information indicating an estimation result of the personality of the estimation target person.

8. The non-transitory computer-readable storage medium according to claim 7, wherein;
the acquiring information indicating the eye closing-related index acquires measurement information indicating at least one of an eye potential of the estimation target person and a captured image including an eyeball site, and obtains the eye closing-related index of the estimation target person on a basis of the acquired measurement information.

9. The non-transitory computer-readable storage medium according to claim 7, wherein;
the acquiring information indicating the eye closing-related index acquires an average blinking frequency of the estimation target person as the eye closing-related index.

10. The non-transitory computer-readable storage medium according to claim 7, wherein:
the acquiring information indicating the movement acquires information indicating a frequency of the movement of the eyeball or the head as the information indicating the movement of the eyeball or the head of the estimation target person.

11. The non-transitory computer-readable storage medium according to claim 7, wherein:
the estimating the personality estimates openness as the personality of the estimation target person.
